(19) United States
(12) Patent Application Publication
(10) Pub. No.: US 2004/0073261 A1
(43) Pub. Date: Apr. 15, 2004

(54) Methods and systems for treating ventricular fibrillation

(76) Inventor: Mark W. Kroll, Simi Valley, CA (US)

Correspondence Address: PACESETTER, INC., 15900 Valley View Court, Sylmar, CA, 91392-9221, US

(21) Appl. No.: 10/268,298

(22) Filed: Oct. 9, 2002

(51) Int. Cl. A61N 1/39

(52) U.S. Cl. 607/5

(57) ABSTRACT

Various embodiments provide methods and devices (e.g. ICDs) for treating ventricular fibrillation. Defibrillation therapy can be administered in which at least a first shock is administered by an electrode that is implanted in the right ventricle. The first shock need not use an electrode that resides within or near the atria (e.g. an SVC coil), thus reducing the risk of atrial fibrillation that can be brought on by administering shocks from within the atria. If the first shock (or subsequent shocks originating from within the right ventricle) is unsuccessful, then the ICD device can automatically connect an additional electrode (e.g. an electrode that resides within or near the right atrium) and deliver additional shocks to attempt to treat the ventricular fibrillation. In this manner, the risk of atrial fibrillation is initially lowered, yet there is no meaningful reduction in the ability to defibrillate the patient.

### FIELD OF THE INVENTION

The present invention generally relates to implantable devices, such as implantable stimulation devices and the like. In particular, the invention pertains to methods and systems for treating ventricular fibrillation.

### BACKGROUND OF THE INVENTION

Ventricular fibrillation is a type of cardiac arrhythmia characterized by rapid, unsynchronized quivering of the ventricles. Ventricular fibrillation is typically fatal if not corrected within minutes. Some types of implantable stimulation devices, such as implantable cardioverter defibrillators (ICDs) are configured to administer cardiac stimulation therapy that is directed to correcting arrhythmias such as ventricular fibrillation. If the heartbeat gets too fast, the ICD will stimulate the heart to restore a normal rhythm. This is known as antitachycardia pacing. In cases where the heartbeat is so rapid that the person may die (i.e. ventricular fibrillation), the ICD will also give an electric shock to “reset” the heartbeat. Electric shocks to treat ventricular fibrillation typically range from a moderate energy level to a high energy level, e.g. 10-40 joules.

ICDs typically include multiple electrodes that are implanted in different regions of the heart. For example, electrodes can be implanted in both the ventricles and atria. There has been somewhat of a debate among industry professionals as to whether one needs an electrode in or near the right atrium for treating ventricular defibrillation. That is, patients who are likely to experience ventricular fibrillation clearly need an electrode in the right ventricle for administering therapeutic shocks to correct ventricular fibrillation. It is less clear, and hence debatable, whether a right atrial or superior vena cava (SVC) electrode is absolutely necessary for purposes of treating ventricular fibrillation. Those in favor of using an SVC electrode in combination with the right ventricular electrode to treat ventricular fibrillation argue that the increased wave front that propagates across the myocardium is desirable insofar as increasing the likelihood of successful treatment.

Using an additional SVC electrode can, however, have undesirable effects. Specifically, it is common knowledge within the industry that moderate current or energy levels within a heart's chamber tend to induce fibrillation. This can be understood and appreciated using a concept known as the “zone of vulnerability”. That is, shocks administered at and above certain energy levels tend to defibrillate a patient who is experiencing fibrillation. However, below that energy level and within some mid-level range of energies (i.e. the zone of vulnerability), shocks will tend to cause fibrillation. Below this mid-level range, stimulation energies typically define pacing pulses that have no lasting effect.

By using a right atrial electrode to administer defibrillation shocks, energy levels within a patient's zone of vulnerability may actually be provided within the atria, particularly in the back area of the atria. This being the case, the risk of inducing atrial fibrillation is thus increased. If atrial fibrillation is induced, though not life threatening, it may eventually have to be treated.

It would be desirable to be able to adequately treat ventricular fibrillation, without initially increasing a patient's risk of atrial fibrillation.

Accordingly, this invention arose out of concerns associated with providing improved methods and systems for treating ventricular fibrillation.

### SUMMARY

Various embodiments provide methods and devices (e.g. ICDs) for treating ventricular fibrillation. Defibrillation therapy can be administered in which at least a first shock is administered by an electrode that is implanted in the right ventricle with the second electrode being the pulse generator housing. The first shock need not use an electrode that resides within or near the atria (e.g. an SVC coil), thus reducing the risk of atrial fibrillation that can be brought on by administering shocks from within the atria.

If the first shock (or subsequent shocks originating from within the right ventricle) is unsuccessful, then the ICD device can automatically connect an additional electrode (e.g. an electrode that resides within or near the right atrium) and deliver additional shocks to attempt to treat the ventricular fibrillation. In this manner, the risk of atrial fibrillation is initially lowered, yet there is no meaningful reduction in the ability to defibrillate the patient.

Some embodiments can increase the shock energy of one or more of any required additional shocks and administer the additional shocks using the new electrode configuration. Yet other embodiments can increase the shock energy of the shocks administered by a first electrode configuration and then incorporate a new electrode configuration when the shock energy satisfies some threshold relationship (e.g. exceeds some defined value). Still further embodiments can automatically switch to the new electrode configuration after the patient has been shocked a predefined number of times (e.g. two times).

### BRIEF DESCRIPTION OF THE DRAWINGS

The following description is of the best mode presently contemplated for practicing the invention. This description is not to be taken in a limiting sense but is made merely for the purpose of describing the general principles of the invention. The scope of the invention should be ascertained with reference to the issued claims.

FIG. 1 is a simplified diagram illustrating an implantable stimulation device in electrical communication with at least three leads implanted into a patient's heart for delivering multi-chamber stimulation and shock therapy.

FIG. 2 is a functional block diagram of a multi-chamber implantable stimulation device illustrating exemplary basic elements of a stimulation device which can provide cardioversion, defibrillation and/or pacing stimulation in up to four chambers of the heart.

FIG. 3 is a flow diagram that describes steps in a method in accordance with one embodiment.

FIG. 4 is a flow diagram that describes steps in a method in accordance with one embodiment.

FIG. 5 is a flow diagram that describes steps in a method in accordance with one embodiment.

FIG. 6 is a flow diagram that describes steps in a method in accordance with one embodiment.

### DETAILED DESCRIPTION

The following description is of the best mode presently contemplated for practicing the invention. This description is not to be taken in a limiting sense but is made merely for the purpose of describing the general principles of the invention. The scope of the invention should be ascertained with reference to the issued claims. In the description of the invention that follows, like numerals or reference designators will be used to refer to like parts or elements throughout.

### Overview

In the embodiments described below, various methods for treating ventricular fibrillation are described. The methods can be implemented in connection with an ICD device that can be programmed to provide defibrillation therapy in which at least the first shock does not use an electrode that resides within or near the atria (e.g. an SVC coil). That is, the initial shock (and, in some embodiments, one or more subsequent shocks) is administered via an electrode implanted within the right ventricle and an electrode in the device housing, thus reducing the risk of atrial fibrillation that can be brought on by administering shocks from within the atria.

If the first shock (or subsequent shocks originating from within the right ventricle) is unsuccessful, then the ICD device can automatically connect an additional electrode (e.g. the SVC coil) and deliver additional shocks to attempt to treat the ventricular fibrillation.

In this manner, the risk of atrial fibrillation is initially lowered, yet there is no meaningful reduction in the ability to defibrillate the patient.

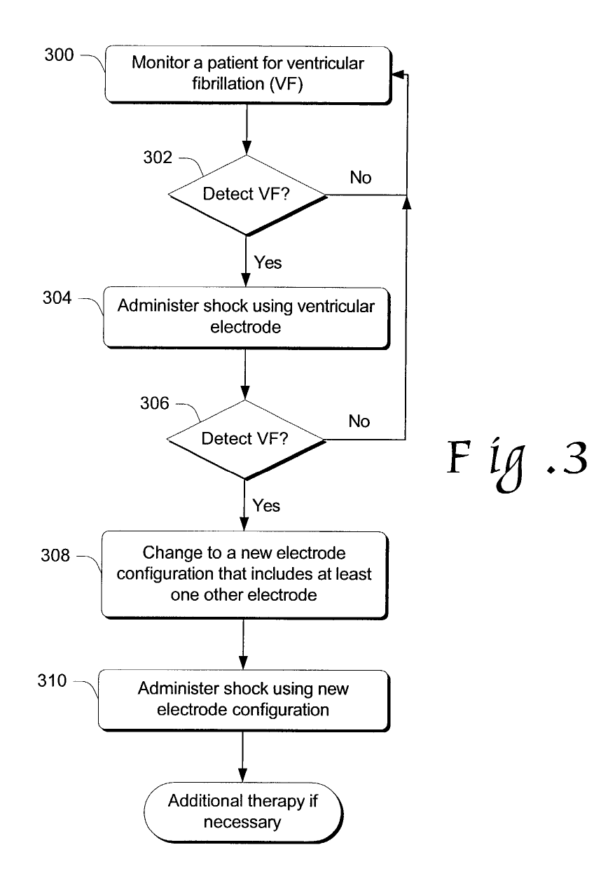

FIG. 3 is a flow diagram that describes steps in a method in accordance with one or more embodiments. The method can be implemented in connection with any suitably configured or configurable implantable stimulation device. One such device is described immediately below in the section entitled “Exemplary Stimulation Device”.

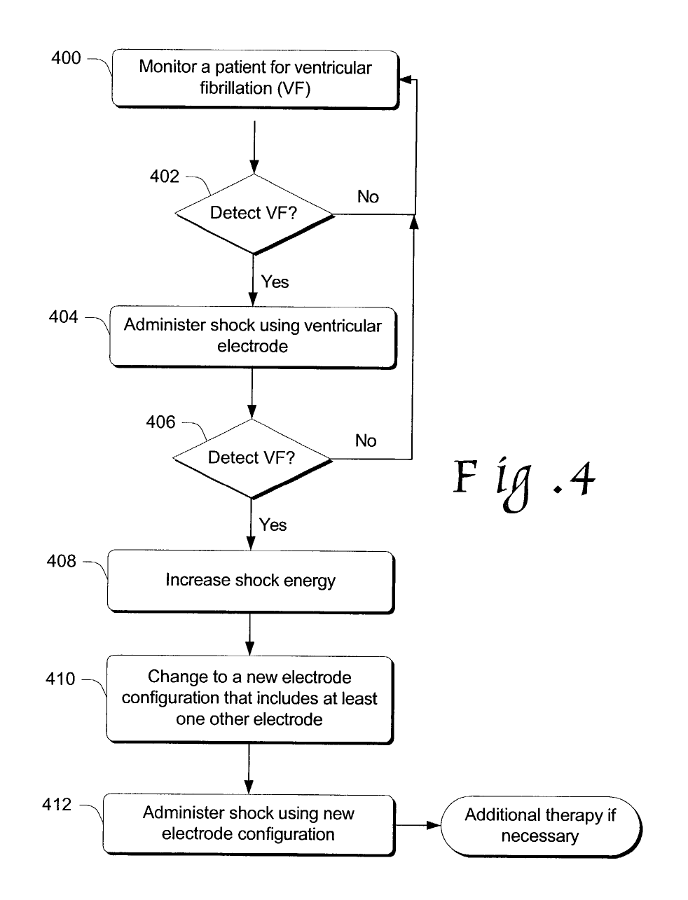

Step 300 monitors a patient for ventricular fibrillation. This step can be implemented using any suitable known techniques for ventricular fibrillation monitoring. Step 302 determines whether the patient is experiencing ventricular fibrillation. If not, the method returns to step 300 and continues to monitor the patient. If, on the other hand, step 302 detects ventricular fibrillation, then step 304 administers at least a first shock using an electrode configuration that comprises an implanted ventricular electrode and the pulse generator housing. The ventricular electrode can comprise any suitable electrode. In one implementation, the electrode comprises a coil electrode. Examples of ventricular electrodes are described in the section below. After administering the first shock, step 306 determines whether the patient is still experiencing ventricular fibrillation. If the ventricular fibrillation has been successfully treated, then the method can return to step 300 to continue to monitor the patient for subsequent episodes of ventricular fibrillation. If, on the other hand, the patient is still experiencing ventricular fibrillation, then step 308 changes to a new electrode configuration that includes at least one other electrode. In this example the other electrode can comprise an electrode that is implanted within or near the atria and, more specifically, the right atrium. Any suitable electrodes can be used. In but one example, this newly incorporated electrode comprises an SVC electrode. Alternately or additionally, before changing the electrode configuration at step 308, and responsive to detecting that the patient is continuing to experience ventricular fibrillation (at step 306), additional shocks at the same or different energy levels can be administered as by looping back to step 304. Specific examples are provided below in connection with FIGS. 4-6.

After step 308 changes to the new electrode configuration, step 310 administers at least one additional shock using the new electrode configuration. This step can also include adjusting the energy level of the shock to a different, higher level than the immediately preceding shock.

After step 310, additional cardiac therapy can be administered in the event that the ventricular fibrillation has not been successfully treated. This can include administering additional shock using the electrode configuration of step 308 at the same energy level or higher.

### Exemplary Stimulation Device

The following description sets forth but one exemplary stimulation device that is capable of being used in connection with the various embodiments that are described below. It is to be appreciated and understood that other stimulation devices, including those that are not necessarily implantable, can be used and that the description below is given, in its specific context, to assist the reader in understanding, with more clarity, the inventive embodiments described herein.

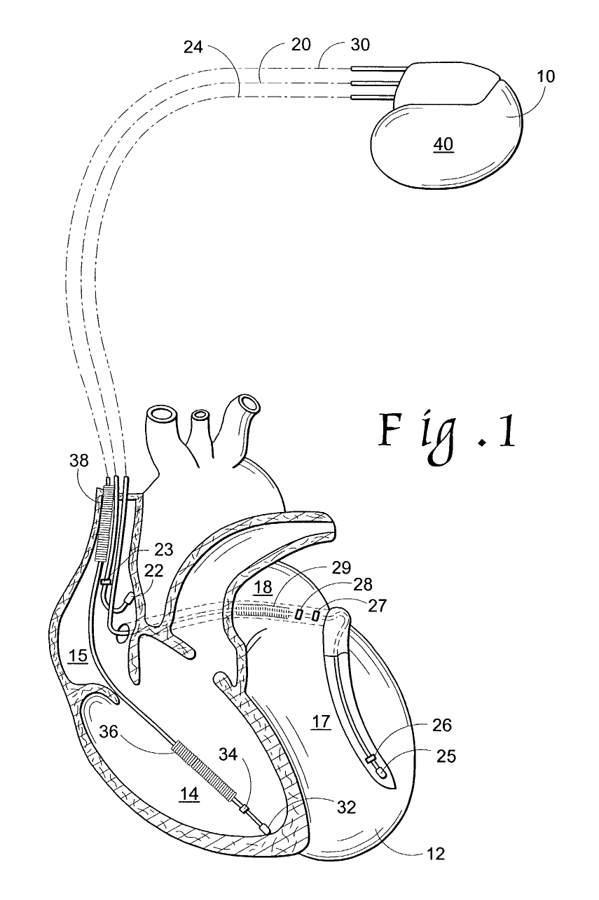

FIG. 1 illustrates a stimulation device 10 in electrical communication with a patient's heart 12 suitable for delivering multi-chamber stimulation and shock therapy. The portions of the heart 10 illustrated include the right ventricle 14, the right atrium 15, the left ventricle 17, and the left atrium 18. As used herein, the left-side of the heart is meant to denote the portions of the heart encompassing the left ventricle 17 and the left atrium 18 and those portions of the coronary sinus, great cardiac vein, and its associated tributaries, which are adjacent the left atrium and left ventricle. Device 10 is desirably configured to administer standard cardiac stimulation therapy as well as stimulation therapy in accordance with the embodiments described herein.

To sense atrial cardiac signals and to provide right atrial chamber stimulation therapy, the stimulation device 10 is coupled to an implantable right atrial lead 20 having at least an atrial tip electrode 22, and preferably a right atrial ring electrode 23, which typically is implanted in the patient's right atrial appendage.

To sense left atrial and ventricular cardiac signals and to provide left-chamber pacing therapy, the stimulation device 10 is coupled to a “coronary sinus” lead 24 designed for placement in the “coronary sinus region” via the coronary sinus so as to place one or more distal electrodes adjacent to the left ventricle 17 and one or more proximal electrodes adjacent to the left atrium 18. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus.

Accordingly, the coronary sinus lead 24 is designed to receive atrial and ventricular cardiac signals and to deliver left ventricular pacing therapy using, for example, a left ventricular tip electrode 25 and a left ventricular ring electrode 26; left atrial pacing therapy using, for example, a first and second left atrial ring electrode, 27 and 28; and shocking therapy using at least a left atrial coil electrode 29.

The stimulation device 10 is also shown in electrical communication with the patient's heart 12 by way of an implantable right ventricular lead 30 having a right ventricular tip electrode 32, a right ventricular ring electrode 34, a right ventricular (RV) coil electrode 36, and an SVC coil electrode 38. Typically, the right ventricular lead 30 is transvenously inserted into the heart 12 so as to place the right ventricular tip electrode 32 in the right ventricular apex so that the RV coil electrode 36 will be positioned in the right ventricle and the SVC coil electrode 38 will be positioned in the superior vena cava. Accordingly, the right ventricular lead 30 is capable of receiving cardiac signals, and delivering stimulation in the form of pacing and shock therapy to the right ventricle 14.

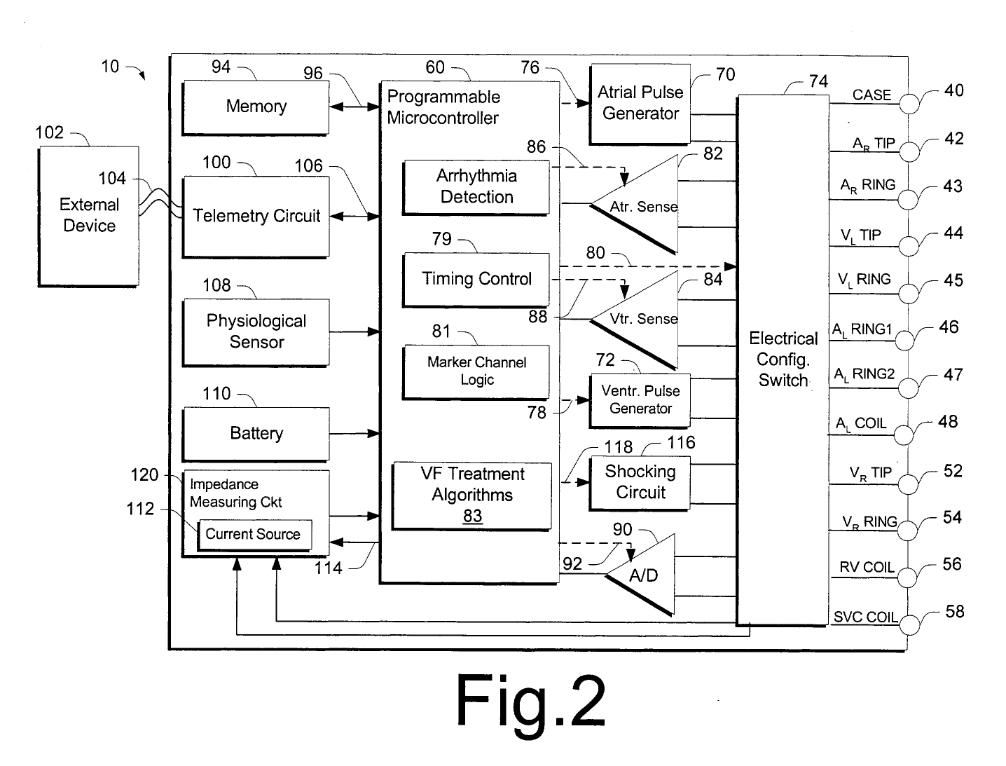

FIG. 2 illustrates a simplified block diagram of the multi-chamber implantable stimulation device 10, which is capable of treating both fast and slow arrhythmias with stimulation therapy, including cardioversion, defibrillation, and pacing stimulation. While a particular multi-chamber device is shown, this is for illustration purposes only, and one of skill in the art could readily duplicate, eliminate or disable the appropriate circuitry in any desired combination to provide a device capable of treating the appropriate chamber(s) with cardioversion, defibrillation and/or pacing stimulation. In addition, it will be appreciated and understood that various processing steps about to be described can be implemented in the form of software instructions that are resident on a computer-readable media that is located on the stimulation device. Accordingly, aspects of the invention described herein extend to all forms of computer-readable media, whether on the stimulation device or not, when such media contains instructions that, when executed by one or more processors, implement the methods described herein.

The stimulation device 10 includes a housing 40 which is often referred to as “can”, “case”, “pulse generator housing” or “case electrode”, and which may be programmably selected to act as the return electrode for all “unipolar” modes. The housing 40 may further be used as a return electrode alone or in combination with one or more of the coil electrodes 29, 36, or 38, for shocking purposes.

The housing 40 further includes a connector (not shown) having a plurality of terminals, 42, 43, 44, 45, 46, 47, 48, 52, 54, 56, and 58 (shown schematically and, for convenience, the names of the electrodes to which they are connected are shown next to the terminals). While it is recognized that current devices are limited to the number of terminals due to International Standards, one of skill in the art could readily eliminate some of the terminals/electrodes to fit in the existing device configurations and permit programmability to select which terminals connect to which electrodes. However, in the near future, the standards may change to permit multi-polar in-line connectors, and multiple feedthroughs connectors could readily be manufactured to accommodate the configuration shown in FIG. 2.

As such, to achieve right atrial sensing and pacing, the connector includes at least a right atrial tip terminal 42 and a right atrial ring terminal 43, adapted for connection to the atrial tip electrode and ring electrodes 22 and 23, respectively.

To achieve left chamber sensing, pacing and/or shocking, the connector includes at least a left ventricular tip terminal 44, a left ventricular ring electrode 45, a first left atrial ring terminal 46, a second left atrial ring terminal 47, and a left atrial shocking terminal 48, which are adapted for connection to the left ventricular tip electrode 25, left ventricular ring 26, the first left atrial tip electrode 27, the second left atrial ring electrode 28, and the left atrial coil electrode 29, respectively.

To support right chamber sensing, pacing and/or shocking, the connector further includes a right ventricular tip terminal 52, a right ventricular ring terminal 54, a right ventricular (RV) shocking terminal 56, and an SVC shocking terminal 58, which are adapted for connection to the right ventricular tip electrode 32, right ventricular ring electrode 34, the RV coil electrode 36, and the SVC coil electrode 38, respectively.

At the core of the stimulation device 10 is a programmable microcontroller or microprocessor 60 that controls the various modes of stimulation therapy. As is well known in the art, the microcontroller 60 typically includes a microprocessor, or equivalent control circuitry, designed specifically for controlling the delivery of stimulation therapy, and may further include RAM or ROM memory, logic and timing circuitry, state machine circuitry, and I/O circuitry. Typically, the microcontroller 60 includes the ability to process or monitor input signals (data) as controlled by a program code stored in a designated block of memory. The details of the design and operation of the microcontroller 60 are not critical to the present invention. Rather, any suitable microcontroller 60 may be used that carries out the functions described herein. The use of microprocessor-based control circuits for performing timing and data analysis functions are well known in the art.

As shown in FIG. 2, an atrial pulse generator 70 and a ventricular pulse generator 72 generate pacing stimulation pulses for delivery by the right atrial lead 20, the right ventricular lead 30, and/or the coronary sinus lead 24 via a switch bank 74. It is understood that in order to provide stimulation therapy in each of the four chambers of the heart, the atrial pulse generator 70 and the ventricular pulse generator 72 may include dedicated, independent pulse generators, multiplexed pulse generators, or shared pulse generators. The atrial pulse generator 70 and the ventricular pulse generator 72 are controlled by the microcontroller 60 via appropriate control signals 76 and 78, respectively, to trigger or inhibit the stimulation pulses.

The microcontroller 60 further includes timing control circuitry 79 which is used to control the timing of such stimulation pulses (e.g., pacing rate, atrio-ventricular (AV) delay, atrial interconduction (A-A) delay, or ventricular interconduction (V-V) delay, etc.), as well as to keep track of the timing of refractory periods, PVARP intervals, noise detection windows, evoked response windows, alert intervals, marker channel timing (via marker channel logic 81), etc., which is well known in the art.

In one embodiment, the microcontroller can be programmed with one or more ventricular fibrillation treatment algorithms 83, in accordance with the described embodiments. The ventricular fibrillation treatment algorithm(s) operate to attempt to avoid (or at least reduce the likelihood of) inducing atrial fibrillation when shock therapy for treating ventricular fibrillation is administered.

The switch bank 74 includes a plurality of switches for connecting the desired electrodes to the appropriate I/O circuits, thereby providing complete electrode programmability. Accordingly, the switch bank 74, in response to a control signal 80 from the microcontroller 60, determines the polarity of the stimulation pulses (e.g. unipolar, bipolar, combipolar, etc.) and various shocking vectors by selectively closing the appropriate combination of switches.

Atrial sensing circuits 82 and ventricular sensing circuits 84 may also be selectively coupled to the right atrial lead 20, coronary sinus lead 24, and the right ventricular lead 30, through the switch bank 74, for detecting the presence of cardiac activity in each of the four chambers of the heart. Accordingly, the atrial and ventricular sensing circuits 82 and 84 may include dedicated sense amplifiers, multiplexed amplifiers, or shared amplifiers. The switch bank 74 determines the “sensing polarity” of the cardiac signal by selectively closing the appropriate switches. In this way, the clinician may program the sensing polarity independent of the stimulation polarity.

The atrial sensing circuit 82 or the ventricular sensing circuit 84 preferably employ one or more low power, precision amplifiers with programmable gain and/or automatic gain control, bandpass filtering, and a threshold detection circuit, to selectively sense the cardiac signal of interest. The automatic gain control enables the stimulation device 10 to deal effectively with the difficult problem of sensing the low amplitude signal characteristics of atrial or ventricular fibrillation. The outputs of the atrial and ventricular sensing circuits, 82 and 84, are connected to the microcontroller 60 for triggering or inhibiting the atrial and ventricular pulse generators, 70 and 72, respectively, in a demand fashion, in response to the absence or presence of cardiac activity, respectively, in the appropriate chambers of the heart.

For arrhythmia detection, the stimulation device 10 utilizes the atrial and ventricular sensing circuits, 82 and 84, to sense cardiac signals for determining whether a rhythm is physiologic or pathologic. As used herein “sensing” is reserved for the noting of an electrical signal, and “detection” is the processing of these sensed signals and noting the presence of an arrhythmia. The timing intervals between sensed events (e.g. P-waves, R-waves, and depolarization signals associated with fibrillation which are sometimes referred to as “F-waves” or “Fib-waves”) are then classified by the microcontroller 60 by comparing them to a predefined rate zone limit (e.g. bradycardia, normal, low rate VT, high rate VT, and fibrillation rate zones) and various other characteristics (e.g. sudden onset, stability, physiologic sensors, and morphology, etc.) in order to determine the type of remedial therapy that is needed (e.g. bradycardia pacing, anti-tachycardia pacing, cardioversion shocks or defibrillation shocks, collectively referred to as “tiered therapy”).

Cardiac signals are also applied to the inputs of an analog-to-digital (A/D) data acquisition system 90. The data acquisition system 90 is configured to acquire intracardiac electrogram signals, convert the raw analog data into digital signals, and store the digital signals for later processing and/or telemetric transmission to an external device 102. The data acquisition system 90 is coupled to the right atrial lead 20, the coronary sinus lead 24, and the right ventricular lead 30 through the switch bank 74 to sample cardiac signals across any pair of desired electrodes.

The microcontroller 60 is further coupled to a memory 94 by a suitable data/address bus 96, wherein the programmable operating parameters used by the microcontroller 60 are stored and modified, as required, in order to customize the operation of the stimulation device 10 to suit the needs of a particular patient. Such operating parameters define, for example, pacing pulse amplitude, pulse duration, electrode polarity, rate, sensitivity, automatic features, arrhythmia detection criteria, and the amplitude, waveshape and vector of each shocking pulse to be delivered to the patient's heart 12 within each respective tier of therapy.

Advantageously, the operating parameters of the stimulation device 10 may be non-invasively programmed into the memory 94 through a telemetry circuit 100 in telemetric communication with the external device 102, such as a programmer, transtelephonic transceiver, or a diagnostic system analyzer. The telemetry circuit 100 is activated by the microcontroller 60 by a control signal 106. The telemetry circuit 100 advantageously allows intracardiac electrograms and status information relating to the operation of the stimulation device 10 (as contained in the microcontroller 60 or memory 94) to be sent to the external device 102 through the established communication link 104.

The stimulation device 10 can further include one or more physiologic sensors 108. Some physiologic sensors are referred to as “rate-responsive” sensors because they are typically used to adjust pacing stimulation rate according to the exercise state of the patient. However, physiological sensors 108 may further be used to detect changes in cardiac output, changes in the physiological condition of the heart, patient activity, or diurnal changes in activity (e.g. detecting sleep and wake states). A physiological parameter of the heart, which may be measured to optimize such pacing and to indicate when such pacing may be inhibited or terminated is the stroke volume of the heart. Accordingly, the microcontroller 60 responds by adjusting the various pacing parameters (such as rate, AV Delay, A-A Delay, V-V Delay, etc.) at which the atrial and ventricular pulse generators, 70 and 72, generate stimulation pulses. A common type of rate responsive sensor is an activity sensor, such as an accelerometer or a piezoelectric crystal, which is mounted within the housing 40 of the stimulation device 10. Other types of physiologic sensors are also known, for example, sensors which sense the oxygen content of blood, respiration rate and/or minute ventilation, pH of blood, ventricular gradient, etc.

The stimulation device 10 additionally includes a power source such as a battery 110 that provides operating power to all the circuits shown in FIG. 2. For the stimulation device 10, which employs shocking therapy, the battery 110 is capable of operating at low current drains for long periods of time, and also be capable of providing high-current pulses (for capacitor charging) when the patient requires a shock pulse. The battery 110 preferably has a predictable discharge characteristic so that elective replacement time can be detected. Accordingly, the stimulation device 10 can employ lithium/silver vanadium oxide batteries.

It can be a primary function of the stimulation device 10 to operate as an implantable cardioverter/defibrillator (ICD) device. That is, it can detect the occurrence of an arrhythmia, and automatically apply an appropriate electrical shock therapy to the heart aimed at terminating the detected arrhythmia. To this end, the microcontroller 60 further controls a shocking circuit 116 by way of a control signal 118. The shocking circuit 116 generates shocking pulses of low (up to 0.5 joules), moderate (0.5-10 joules), or high (11 to 40 joules) energy, as controlled by the microcontroller 60. Such shocking pulses are applied to the patient's heart through at least two shocking electrodes, and as shown in this embodiment, selected from the left atrial coil electrode 29, the RV coil electrode 36, and/or the SVC coil electrode 38 (FIG. 1). As noted above, the housing 40 may act as an active electrode in combination with the RV electrode 36, or as part of a split electrical vector using the SVC coil electrode 38 or the left atrial coil electrode 29 (i.e., using the RV electrode as the common electrode).

Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 5-40 joules), delivered asynchronously (since R-waves may be too disorganized), and pertaining exclusively to the treatment of fibrillation. Accordingly, the microcontroller 60 is capable of controlling the synchronous or asynchronous delivery of the shocking pulses.

As further shown in FIG. 2, the stimulation device 10 can have an impedance measuring circuit 120 including an impedance measuring current source 112 and a voltage measuring circuit 90 (shown in FIG. 2 as an A/D converter), which can be enabled by the microcontroller 60 by a control signal 114 for providing stroke volume measurements of the heart. The current source 112 can provide an alternating or pulsed excitation current. The voltage measuring circuitry 90 may also take the form of, for example, a differential amplifier.

The uses for an impedance measuring circuit 120 include, but are not limited to, lead impedance surveillance during the acute and chronic phases for proper lead positioning or dislodgment; detecting operable electrodes and automatically switching to an operable pair if dislodgment occurs; measuring a respiration parameter (for example, tidal volume, respiration rate, minute ventilation or volume, abnormal or periodic breathing); measuring thoracic impedance for determining shock thresholds and shock timing (corresponding to the diastolic time); detecting when the device has been implanted; measuring a cardiac parameter (such as, stroke volume, wall thickness, left ventricular volume, etc.); and detecting the opening of the valves, etc. The impedance measuring circuit 120 can be advantageously coupled to the switch bank 74 so that any desired electrode can be used. Impedance may also be useful in verifying hemodynamic collapse to confirm that ATP has failed and/or VF has begun.

The microcontroller 60 is coupled to the voltage measuring circuit 90 and the current source 112 for receiving a magnitude of the established current and a magnitude of the monitored voltage. The microcontroller 60, operating under program instructions, divides the magnitude of the monitored or measured voltage by the magnitude of the established current to determine an impedance value. Once the impedance signals are determined, they may be delivered to the memory 94 for storage and later retrieved by the microcontroller 60 for therapy adjustment or telemetry transmission. The telemetry circuitry receives the impedance values from the microcontroller 60 and transmits them to the external programmer. The impedance value may then be monitored by the patient's physician to enable the physician to track the patient's condition.

The impedance measuring circuit 120 is advantageously coupled to the switch bank 74 so that any desired electrode may be used. The current source 112 may be programmably configured between a desired pair of electrodes, and the voltage measuring circuit 90 may be programmably configured between the same or preferably a different pair of electrodes.

### Treating VF VF Increasing the Shock Energy and Changing the Electrode Configuration

FIG. 4 is a flow diagram that describes steps in a method in accordance with one or more embodiments. The method can be implemented in connection with any suitably configured or configurable implantable stimulation device. One such device is described above in the section entitled “Exemplary Stimulation Device”. The method about to be described is directed to treating ventricular fibrillation by first attempting to defibrillate a patient by shocking the patient using a ventricular electrode. If defibrillation attempts are unsuccessful, then the shock energy can be increased and defibrillation can be attempted using a new electrode configuration.

Step 400 monitors a patient for ventricular fibrillation. This step can be implemented using any suitable known techniques for ventricular fibrillation monitoring. Step 402 determines whether the patient is experiencing ventricular fibrillation. If not, the method returns to step 400 and continues to monitor the patient. If, on the other hand, step 402 detects ventricular fibrillation, then step 404 administers at least a first shock using an electrode configuration that comprises an implanted ventricular electrode. The ventricular electrode can comprise any suitable electrode. In one implementation, the electrode comprises a coil electrode. Examples of ventricular electrodes are described in the section above. The first shock is administered at a first energy level—say, for example, 10 joules.

After administering the first shock, step 406 determines whether the patient is still experiencing ventricular fibrillation. If the ventricular fibrillation has been successfully treated, then the method can return to step 400 to continue to monitor the patient for subsequent episodes of ventricular fibrillation. If, on the other hand, the patient is still experiencing ventricular fibrillation, then step 408 can increase the shock energy level and step 410 changes to a new electrode configuration that includes at least one other electrode. In this example the other electrode can comprise an electrode that is implanted within or near the atria and, more specifically, the right atrium. Any suitable electrodes can be used. In but one example, this newly incorporated electrode comprises an SVC electrode. Alternately or additionally, before changing the electrode configuration at step 410, and responsive to detecting that the patient is continuing to experience ventricular fibrillation (at step 406), additional shocks at the same or different energy levels can be administered as by looping back to step 404.

After step 410 changes to the new electrode configuration, step 412 administers at least one additional shock using the new electrode configuration at the increased energy level.

For example, if a first initial shock is delivered at 10 joules and the patient continues ventricular fibrillation, then the shock energy level might be increased to 15 joules and a new electrode configuration that includes an SVC coil in the right atrium can be used to administer the next shock.

After step 412, additional cardiac therapy can be administered in the event that the ventricular fibrillation has not been successfully treated. This can include administering additional shocks using the electrode configuration of step 410 at the same energy level or higher.

### Treating VF by Increasing the Shock Energy and Changing the Electrode Configuration at a Predetermined Energy Threshold

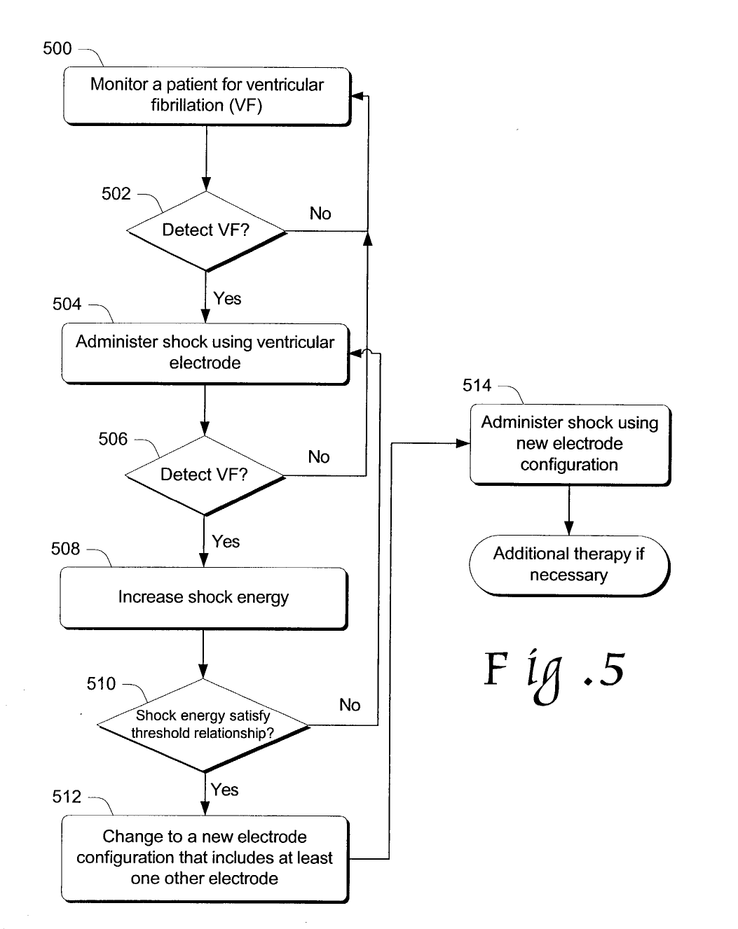

FIG. 5 is a flow diagram that describes steps in a method in accordance with one or more embodiments. The method can be implemented in connection with any suitably configured or configurable implantable stimulation device. One such device is described above in the section entitled “Exemplary Stimulation Device”. The method about to be described is directed to treating ventricular fibrillation by first attempting to defibrillate a patient by shocking the patient using a ventricular electrode. If defibrillation attempts are unsuccessful, then the shock energy can be increased and defibrillation can be attempted again. When the shock energy level satisfies a certain threshold relationship, then a new electrode configuration can be used to attempt to defibrillate the patient.

Step 500 monitors a patient for ventricular fibrillation. This step can be implemented using any suitable known techniques for ventricular fibrillation monitoring. Step 502 determines whether the patient is experiencing ventricular fibrillation. If not, the method returns to step 500 and continues to monitor the patient. If, on the other hand, step 502 detects ventricular fibrillation, then step 504 administers at least a first shock using an electrode configuration that comprises an implanted ventricular electrode. The ventricular electrode can comprise any suitable electrode. In one implementation, the electrode comprises a coil electrode. Examples of ventricular electrodes are described in the section above. The first shock is administered at a first energy level—say, for example, 10 joules.

After administering the first shock, step 506 determines whether the patient is still experiencing ventricular fibrillation. If the ventricular fibrillation has been successfully treated, then the method can return to step 500 to continue to monitor the patient for subsequent episodes of ventricular fibrillation. If, on the other hand, the patient is still experiencing ventricular fibrillation, then step 508 can increase the shock energy level and step 510 determines whether the shock energy level satisfies a threshold relationship. If the shock energy level does not satisfy the threshold relationship, then the method can loop back to step 504 and administer a shock at the new energy level. If, on the other hand, the shock energy level satisfies the threshold relationship, then step 512 changes to a new electrode configuration that includes at least one other electrode. In this example the other electrode can comprise an electrode that is implanted within or near the atria and, more specifically, the right atrium. Any suitable electrodes can be used. In but one example, this newly incorporated electrode comprises an SVC electrode. Alternately or additionally, before changing the electrode configuration at step 512, and responsive to detecting that the patient is continuing to experience ventricular fibrillation (at step 506), additional shocks at the same or different energy levels can be administered as by looping back to step 504.

After step 512 changes to the new electrode configuration, step 514 administers at least one additional shock using the new electrode configuration at the increased energy level.

For example, if a first initial shock is delivered at 10 joules and the patient continues in ventricular fibrillation, then the shock energy level might be increased to 15 joules. If, in this example, the threshold relationship is defined as follows: “Is the shock level set at or above 13 joules?”, then step 510 would evaluate affirmatively and the method would proceed to step 512. If, on the other hand, the threshold relationship is defined as follows: “Is the shock level set at or above 18 joules?”, then step 510 would evaluate negatively and the method would loop back to step 504.

After step 514, additional cardiac therapy can be administered in the event that the ventricular fibrillation has not been successfully treated. This can include administering additional shocks using the electrode configuration of step 512 at the same energy level or higher.

### Treating VF By Changing the Electrode Configuration After a Predetermined Number of Shocks Using a First Electrode Configuration

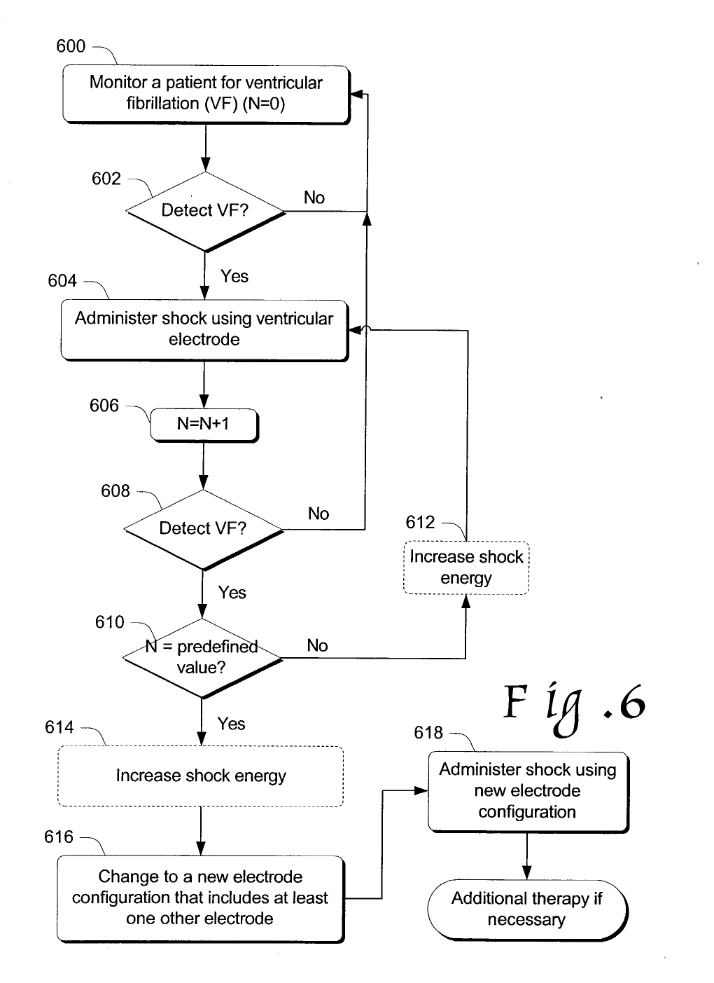

FIG. 6 is a flow diagram that describes steps in a method in accordance with one or more embodiments. The method can be implemented in connection with any suitably configured or configurable implantable stimulation device. One such device is described above in the section entitled “Exemplary Stimulation Device”. The method about to be described is directed to treating ventricular fibrillation by first attempting to defibrillate a patient by shocking the patient using a ventricular electrode. If defibrillation attempts are unsuccessful after a defined number of attempts using the ventricular electrode, then defibrillation can be attempted using a new electrode configuration.

Step 600 monitors a patient for ventricular fibrillation. This step can be implemented using any suitable known techniques for ventricular fibrillation monitoring. Notice also in the decision box for this step that a variable N is defined as N=0. This variable is used as a counter to keep track of how many times a defibrillation shock is administered to a patient for a particular fibrillation episode. Step 602 determines whether the patient is experiencing ventricular fibrillation. If not, the method returns to step 600 and continues to monitor the patient.

If, on the other hand, step 602 detects ventricular fibrillation, then step 604 administers at least a first shock using an electrode configuration that comprises an implanted ventricular electrode. The ventricular electrode can comprise any suitable electrode. In one implementation, the electrode comprises a coil electrode. Examples of ventricular electrodes are described above.

After administering the first shock, step 606 increments N by 1 and step 608 determines whether the patient is still experiencing ventricular fibrillation. If the ventricular fibrillation has been successfully treated, then the method can return to step 600 to continue to monitor the patient for subsequent episodes of ventricular fibrillation. Accordingly, N is set back to 0. If, on the other hand, the patient is still experiencing ventricular fibrillation, then step 610 determines whether the patient has been shocked a predefined number of times. For example, this step determines if N equals a predefined value, e.g. “2”. If the patient has not been shocked a predefined number of times, then the method can loop back to step 604 and administer another shock. Optionally, in looping back to step 604, step 612 can, but need not, increase the shock energy level of the shock so that the patient receives a subsequent shock at a higher energy level.

If, on the other hand, the patient has been shocked a predefined number of times, then, optionally, step 614 can increase the shock energy level and step 616 can change to a new electrode configuration that includes at least one other electrode. In this example the other electrode can comprise an electrode that is implanted within or near the atria and, more specifically, the right atrium. Any suitable electrodes can be used. In but one example, this newly incorporated electrode comprises an SVC electrode.

After step 616 changes to the new electrode configuration, step 618 administers at least one additional shock using the new electrode configuration.

After step 618, additional cardiac therapy can be administered in the event that the ventricular fibrillation has not been successfully treated. This can include administering additional shocks using the electrode configuration of step 616 at the same energy level or higher.

As an example, consider the following. Assume that a patient experiences ventricular fibrillation and an initial shock is administered by a ventricular electrode at say 10 joules. If this initial shock does not bring the patient out of their fibrillation then, according to the above method, the shock energy might then be increased to 15 joules and the patient can again be shocked using only the ventricular electrode. If this does not bring the patient out of their fibrillation, then the shock energy might then be increased to 36 joules and the electrode configured changed to now include an SVC coil in the right atrium. The patient can then be shocked at the higher energy level using the new configuration.

### Shortening Pulse Widths

In accordance with the embodiments described above, when the electrode configuration is changed to the new electrode configuration (as in steps 308 (FIG. 3), 410 (FIG. 4), 512 (FIG. 5), and 616 (FIG. 6)), the pulse width of the subsequently administered shock can be shortened from that of the previous shocks. One reason to do this, particular in embodiments where the right atrial electrode comprises an SVC coil is that the SVC coil significantly lowers the impedance of the system. As a result, shortened pulse widths can improve defibrillation efficacy.

### Conclusion

There can be several advantages associated with the above-described embodiments. First and perhaps foremost is that the described methods can initially attempt to treat the ventricular fibrillation while, at the same time, reduce the chances of inducing atrial fibrillation. If the ventricular fibrillation is successfully treated without using an electrode positioned within or near the atria, then this is all the better for the patient. If, on the other hand, the first initial shock or shocks do not successfully treat the ventricular fibrillation, then a new electrode configuration can be used, albeit with a risk of atrial fibrillation (a condition that is not immediately life threatening).

Additionally, the longevity of the stimulation device can conceivably be extended because there may be instances where a fibrillation is successfully treated without using the full energy potential of the device. Further, patient discomfort can potentially be reduced by using a shock with a lower energy level to hopefully successfully treat ventricular fibrillation. Using lower shock energies can also be advantageous when a shock is administered for inappropriate reasons. For example, there may be a circumstance when the device inappropriately determines that the patient is experiencing ventricular fibrillation due to perhaps noise that is received by the device. In this case, having a first initial shock administered from only a ventricular electrode at a lower energy can help to reduce the patient's discomfort. Other advantages should be apparent to those of skill in the art.

Although the invention has been described in language specific to structural features and/or methodological steps, it is to be understood that the invention defined in the appended claims is not necessarily limited to the specific features or steps described. Rather, the specific features and steps are disclosed as preferred forms of implementing the claimed invention.

What is claimed is:

1. A method comprising:
responsive to a patient experiencing ventricular fibrillation, administering at least one shock using only a right ventricular electrode configuration that comprises a right ventricular electrode and a second electrode that does not include an electrode proximate the patient's right atrium; and
responsive to the patient continuing to experience ventricular fibrillation, administering at least one additional shock using a different electrode configuration that includes at least one electrode proximate the patient's right atrium.

2. The method of claim 1, wherein the act of administering at least one shock comprises administering a first shock and, if necessary, administering a second shock in an event that the patient continues to experience ventricular fibrillation.

3. The method of claim 1, wherein the act of administering at least one additional shock using said different electrode configuration comprises using a configuration that includes the right ventricular electrode.

4. The method of claim 1, wherein the one electrode that is proximate the right atrium comprises a superior vena cava electrode.

5. The method of claim 1, wherein said administering at least one additional shock comprises administering at least one shock having a pulse width that is shorter than at least one previously-administered shock.

6. A stimulation device comprising:
memory;
one or more processors;
instructions in the memory which, when executed by the one or more processors, cause the one or more processors to:
monitor a patient for ventricular fibrillation;
responsive to a patient experiencing ventricular fibrillation, administer at least one shock using an electrode configuration that includes at least one right ventricular electrode but does not include an electrode proximate the patient's right atrium; and
responsive to the patient continuing to experience ventricular fibrillation, administer at least one additional shock using a different electrode configuration that comprises at least one electrode proximate the patient's right atrium.

7. The stimulation device of claim 6, wherein the instructions cause the one or more processors to administer said at least one shock by administering a first shock and, if necessary, administering a second shock in an event that the patient continues to experience ventricular fibrillation.

8. The stimulation device of claim 6, wherein the instructions cause the one or more processors to administer said at least one additional shock using an electrode configuration that includes the right ventricular electrode.

9. A method comprising:
responsive to a patient experiencing ventricular fibrillation, administering at least one shock using a right ventricular electrode configuration that comprises a right ventricular electrode and a pulse generator housing, said at least one shock being administered at a first shock energy level; and
responsive to the patient continuing to experience ventricular fibrillation, administering at least one additional shock using a different electrode configuration that comprises at least the right ventricular electrode and a superior vena cava electrode.

10. The method of claim 9, wherein the act of administering at least one additional shock comprises administering at least one additional shock at a second shock energy level that is different from the first shock energy level.

11. The method of claim 9, wherein the second shock is administered at a shock energy level that is different from the first shock energy level.

12. The method of claim 9, wherein the different electrode configuration includes a pulse generator housing electrode coupled in common with the superior vena cava electrode.

13. The method of claim 9, wherein said administering at least one additional shock comprises administering at least one shock having a pulse width that is shorter than at least one previously-administered shock.

14. A method comprising:
responsive to a patient experiencing ventricular fibrillation, administering at least one shock using a right ventricular electrode configuration that comprises at least one right ventricular electrode, said at least one shock being administered at a first shock energy level;
responsive to the patient continuing to experience ventricular fibrillation, administering one or more additional shocks using the right ventricular electrode configuration at increasingly greater shock energy levels; and
responsive to (a) the patient continuing to experience ventricular fibrillation and (b) a shock energy level that has been or will be used to administer a shock satisfying an energy level threshold relationship, administering at least one additional shock using a different electrode configuration that comprises at least one electrode within the patient's heart that is not a right ventricular electrode.

15. The method of claim 14, wherein said different electrode configuration comprises the right ventricular electrode.

16. The method of claim 15, wherein said at least one electrode that is not a right ventricular electrode comprises a right atrial electrode.

17. The method of claim 15, wherein said at least one electrode that is not a right ventricular electrode comprises a right atrial coil electrode.

18. The method of claim 14, wherein said administering at least one additional shock comprises administering at least one shock having a pulse width that is shorter than at least one previously-administered shock.

19. A method comprising:
responsive to a patient experiencing ventricular fibrillation, administering at least a first shock using only a right ventricular electrode configuration that includes a right ventricular coil electrode and a second electrode that is not proximate a patient's right atrium;
responsive to (a) the patient continuing to experience ventricular fibrillation and (b) administering a predefined number of shocks using only the right ventricular electrode configuration, administering at least one shock using a different electrode configuration that includes at least one electrode proximate the patient's heart that is not a right ventricular electrode.

20. The method of claim 19, wherein said different electrode configuration includes the right ventricular electrode.

21. The method of claim 19, wherein said at least one electrode that is not a right ventricular electrode comprises a superior vena cava electrode.

22. The method of claim 19, wherein all of said shocks define a shock series, at least some individual shocks of the shock series being administered at energy levels that are greater than previously-administered shocks of the shock series.

23. The method of claim 19, wherein said administering at least one additional shock comprises administering at least one shock having a pulse width that is shorter than at least one previously-administered shock.

24. A method comprising:
responsive to a patient experiencing ventricular fibrillation, administering a first shock using only a right ventricular coil electrode and a pulse generator housing electrode, the first shock being administered at a first shock energy level;
responsive to the patient continuing to experience ventricular fibrillation, administering a second shock using only the right ventricular coil electrode and the pulse generator housing electrode, the second shock being administered at a second shock energy level that is greater than the first shock energy level; and
responsive to the patient continuing to experience ventricular fibrillation, administering at least one additional shock using an electrode configuration comprising the right ventricular coil electrode and a superior vena cava coil electrode, said at least one additional shock being administered at a third shock energy level that is greater than the second shock energy level.

25. The method of claim 24, wherein said administering at least one additional shock comprises administering at least one shock having a pulse width that is shorter than at least one previously-administered shock.

26. A stimulation device programmed to implement the method of claim 24.
